(19) United States
(12) Patent Application Publication
(10) Pub. No.: US 2012/0197066 A1
(43) Pub. Date: Aug. 2, 2012

(54) COUPLING APPARATUS

(75) Inventors: Eric William Abel, Dundee (GB); Francis Fiabane, Angus (GB); Duncan Bowyer, Edinburgh (GB)

(73) Assignee: SENTIENT MEDICAL LIMITED, Dundee (GB)

(21) Appl. No.: 13/380,750

(22) PCT Filed: Jun. 24, 2010
(86) PCT No.: PCT/GB10/51045

§ 371 Date: Apr. 17, 2012

(30) Foreign Application Priority Data
Jun. 24, 2009 (GB) 0910908.3

(51) Int. Cl. A61F 11/04; H04R 25/00

(52) U.S. Cl. 600/25

(57) ABSTRACT

Coupling apparatus for coupling an implantable element to the round window membrane. The apparatus comprises engagement means in the form of a clip or a filler material for engaging the bone surface within the round window niche. This supports the apparatus in the region of the round window membrane.

The present invention relates to coupling apparatus, and more particularly to coupling apparatus for coupling a middle ear implant to the round window membrane.

The term “middle ear” refers to the tympanic cavity, located between the external auditory canal and the cochlea.

In a healthy ear, vibrations of the tympanic membrane, or ear drum, which is located at the boundary between the auditory canal and the tympanic cavity, are communicated across the tympanic cavity to the cochlea by a series of three articulated bones known as the ossicular chain.

The ossicular chain comprises three individual ossicles. Namely, the malleus, the incus and the stapes. The malleus is connected between the tympanic membrane and the incus. The incus is in turn connected between the malleus and the stapes. The stapes comprises a footplate portion which is disposed against a membrane which covers an opening to the cochlea, known as the oval window.

Vibrations of the tympanic membrane are thus transmitted by the ossicles to the oval window membrane, to cause pressure variations within the fluid filled cochlea. These pressure variations are accommodated by the presence of a second membrane covered opening, known as the round window, such that the round window membrane vibrates in counter-phase with the oval window membrane.

The term “middle ear implant” refers generally to devices which can be implanted into the tympanic cavity of patients with sensorineural or conductive hearing loss, to improve their hearing.

Sensorineural hearing loss is attributable to defects in the inner ear which reduce its ability to convert vibrational stimulus into neural activity and/or to defects in the parts of the nervous system associated with hearing.

Conductive hearing loss is attributable to defects in the conductive elements of the middle ear, ie the ossicular chain, which prevent the effective conduction of vibrational energy across the middle ear cavity.

In both cases, the patient=s hearing can be improved by amplifying the vibrational stimulus applied to the inner ear by introducing a hearing actuator for actively vibrating one or more elements within the middle ear in response to an external signal from a microphone or other sensor.

In the case of conductive hearing loss, the patient=s hearing can also be improved by replacing or bypassing all or part of the ossicular chain with a prosthesis, which acts as a conductive bridge.

Such devices are collectively referred to as middle ear implants. Implants which conduct vibrational energy across the middle ear, without themselves generating vibrational energy, are referred to as passive implants. Implants which themselves generate vibrational energy are referred to as active implants. Some middle ear implants may comprise both passive and active elements.

A multitude of different middle ear implants, which extend between a variety of different elements within the middle ear, have been developed. Implants which are coupled directly to the stapes footplate have been found to be particularly effective, because this element vibrates the oval window membrane directly, to cause pressure variations in the fluid filled cochlea.

However, it is now recognised that excitation of the round window membrane rather than the oval window membrane may be preferable in some circumstances. For example, where hearing loss is due to a combination of conductive and sensorineural defects, or where the ossicular chain is diseased or badly misshapen, making it difficult to attach an implant to the stapes.

Although the desirability of stimulating the round window is recognised, mechanically coupling an implant to the round window poses problems. In this respect, the bony terrain which surrounds the round window varies unpredictably from patient to patient, and often contains soft regions which are not suitable for receiving fixation screws and the like. Moreover, the bone immediately surrounding the round window forms the wall of the cochlea, and thus can not be drilled into without the risk of damage to the inner ear.

Previous attempts have been made to provide implants which stimulate the round window directly. However, in general, these attempts either require the implant to be mounted to the temporal bone, a significant distance from the round window, or require complex adjustments to the implant and its attachment means during surgery.

It is an object of the present invention to overcome these problems.

According to one aspect of the present invention, there is provided coupling apparatus for coupling an implantable element to the round window membrane, the apparatus comprising engagement means for engaging the bone surface within the round window niche, to support the apparatus in the region of the round window membrane.

The round window niche is a funnel shaped depression in the medial wall of the tympanic cavity. The round window membrane is located at the wider end of the tympanic cavity, whilst the narrower end is defined by bony prominences, including the bony ridge of the subiculum. The precise form varies from person to person. For example, the round window may be aligned with the niche opening, or may be offset to one side relative to the niche opening, such that it is partially or fully obscured by the bony prominances.

By engaging the bone surface within the round window niche, the apparatus of the present invention provides a stable support, close to the round window, for an implantable device such as a hearing actuator or a prosthesis.

The engagement means is preferably configured for engaging location(s) on the bony prominences which define the round window niche.

In preferred embodiments of the present invention, the engagement means is deformable between a first configuration for insertion through the opening of the round window niche, and a second configuration for engaging the bone surface within the niche.

The engagement means is preferably resiliently deformable for insertion through the opening of the round window niche, and for pressing against the bone surface within the round window niche.

More particularly, the engagement means may be resiliently deformable between a first configuration for insertion through the opening of the round window niche, and a second configuration for engaging the bone surface within the niche. That is to say, the engagement means may be resiliently deformed for insertion through the opening of the round window niche, and then released to engage the bone surface within the round window niche.

The coupling apparatus may be formed to have super-elastic properties. The coupling apparatus is preferably at least partially formed of a super-elastic material. The material from which the apparatus is formed is preferably a nickel titanium alloy, such as Nitinol, or some other alloy or polymer or other material with super-elastic properties.

Moreover, the coupling apparatus is preferably configured such that the deflection between its natural or original configuration and said second configuration is sufficiently large, that the engagement means will operate super-elastically, to grip the bone surface within the round window niche. That is to say, the force exerted by the engagement means on the bone surface within the round window niche will be substantially constant over a wide range of deflections. This is desirable because it means that coupling apparatus of a given size can accommodate significant anatomical variation between patients.

The engagement means may comprise a single engagement section, or a plurality of engagement sections for engaging respective locations on the bone surface within the round window niche.

The or each engagement section may comprise a curved section for engaging the bone surface of the round window niche.

The curved section of the or each engagement section may form an outwardly facing concave surface. This is convenient for engaging location(s) on the bony prominences which define the round window niche.

Where the engagement means comprises a single engagement section, this may take the form of a split collar.

Where the engagement means comprises a plurality of engagement sections, these may take the form of two or more engagement arms which extend from a connecting portion.

The engagement means may conveniently comprise two engagement sections, more preferably three engagement sections.

The connecting portion is conveniently positioned for location externally of the round window niche when the engagement means is engaged with the bone surface within the niche.

In general, the plurality of engagement sections are preferably substantially evenly distributed around a central longitudinal axis of the device. That is to say, where there are two engagement sections, these may be separated by angles of approximately 180 degrees, and where there are three engagement sections, these may be separated by angles of approximately 120 degrees.

Where the engagement means comprises one or more resilient engagement sections, the or each engagement section preferably has a substantially elongate form, with a length that is substantially greater than the perpendicular cross section dimension thereof. More preferably, between 10 and 50 times greater.

In preferred embodiments, the coupling apparatus is configured to accommodate a range of anatomical variations in the form of the round window niche and the location of the round window membrane.

In other embodiments, the coupling apparatus may take different forms to accommodate different anatomical variations. For example, the coupling apparatus may be specifically configured to accommodate a variation in which the round window membrane is substantially aligned with the niche opening.

Alternatively, the engagement means may be specifically configured to accommodate a variation in which the round window membrane is offset to one side relative to the niche opening. To accommodate such a variation, one or more of the engagement sections (or a part of the single engagement section) may have a different form and/or orientation than the other engagement section(s) (or part of the engagement section).

In an alternative embodiment of the present invention, the coupling apparatus comprises a tubular portion for location over or around the round window membrane, wherein the engagement means comprises a filler material for at least partially filling the space between an external surface of the tubular portion and the bone surface within the round window niche, to engage said bone surface and hold the tubular portion in position in relation to the round window membrane.

Accordingly, the filler material holds the tubular portion in place, whilst the tubular portion defines a passage or channel through which an implantable element can be inserted, to stimulate the round window membrane.

The filler material is preferably an ionic cement, hydroxyapatite, or other biocompatible filling material.

The coupling apparatus may comprise a mounting part for mounting to an implantable element.

The mounting part of the coupling apparatus is conveniently positioned for location externally of the round window niche, when the engagement means is engaged with the bone surface within the round window niche.

The mounting part may comprise adjustable mounting means for mounting to an implantable element, such that the mounted position of the implantable element in relation to the coupling apparatus is adjustable.

The adjustable mounting means may allow the mounted position of the implantable element to be adjusted longitudinally of the coupling apparatus. Alternatively, or in addition, the adjustable mounting means may allow the orientation of the implantable element relative to the coupling apparatus to be adjusted.

This allows the implantable element to be adjusted to a suitable position once the coupling apparatus is mounted to the round window niche, and thus allows the apparatus to be adjusted to take account of anatomical variations between patients.

The adjustable mounting means may comprise a threaded surface for engaging a correspondingly threaded surface of an implantable element. Alternatively, the adjustable mounting means may comprise one or more elongate recesses or projections for slidably engaging a correspondingly formed portion of an implantable element. Alternatively or in addition, the adjustable mounting means may comprise a rounded projection or opening for rotatably engaging a correspondingly formed projection or opening of an implantable element.

The coupling apparatus of the present invention may include an implantable element, which may form an integral part of the coupling apparatus, or which may be mounted to a mounting part thereof.

The coupling apparatus is preferably configured such that, when the engagement means is engaged with the bone surface within the round window niche, the implantable element extends to or towards the round window membrane, for conveying vibrational energy thereto.

Preferably, the implantable element extends through the opening of the round window niche.

According to a second aspect of the present invention, there is provided a method of coupling an implantable element to the round window membrane, the method comprising:

providing coupling apparatus for coupling an implantable element to the round window membrane; and

engaging said apparatus with the bone surface within the round window niche. The method may further comprise:

deforming a resiliently deformable engagement means of the coupling apparatus into a first configuration;

inserting the engagement means through the opening of the round window niche in said first configuration; and

releasing the engagement means such that the engagement means engages the bone surface within the round window niche.

Alternatively, the method may comprise:

locating a tubular member over or around the round window membrane;

at least partially filling a space between the external surface of the tubular member and the bone surface within the round window niche with a filler material; and

allowing the filler material to set to hold the tubular member in place.

Embodiments of the present invention will now be described with reference to the accompanying drawings in which:

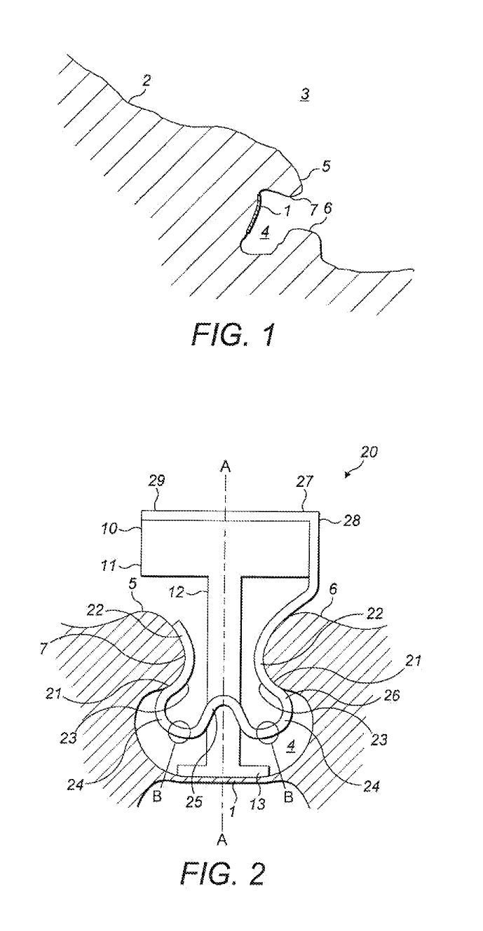

FIG. 1 illustrates the location of the round window niche in the tympanic cavity;

FIG. 2 illustrates a first embodiment of the present invention;

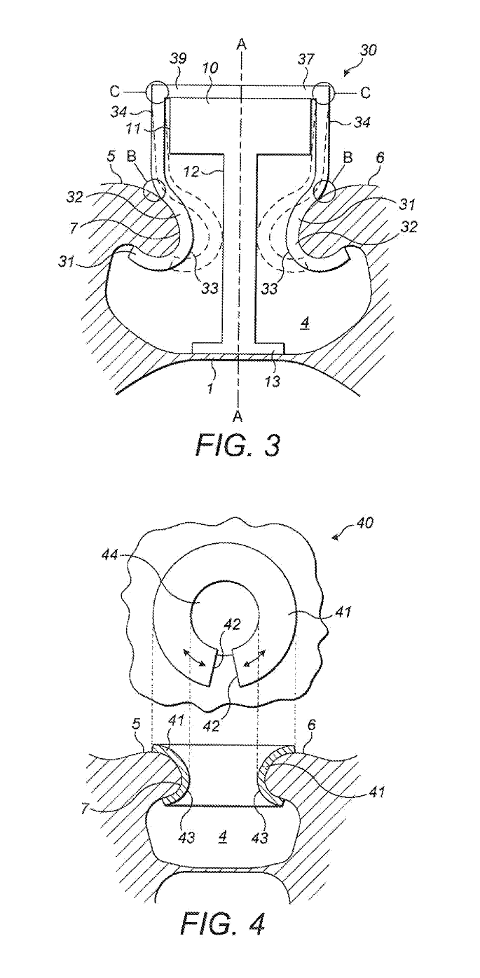

FIG. 3 illustrates a second embodiment of the present invention;

FIG. 4 illustrates a third embodiment of the present invention;

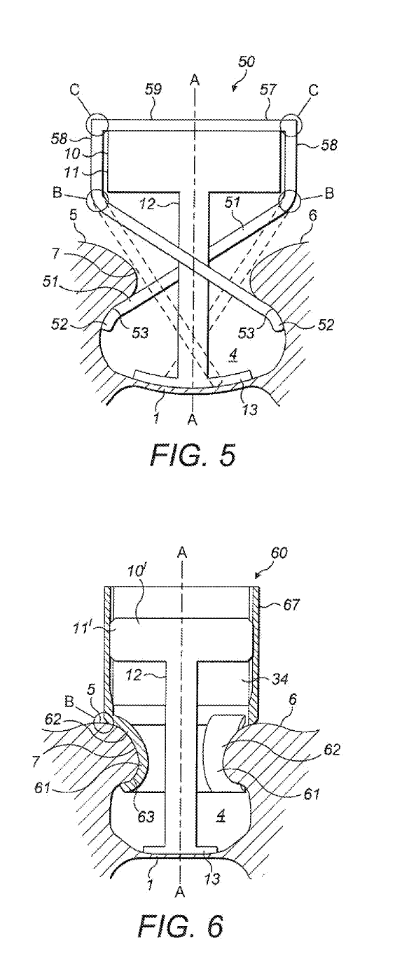

FIG. 5 illustrates a fourth embodiment of the present invention;

FIG. 6 illustrates a fifth embodiment of the present invention;

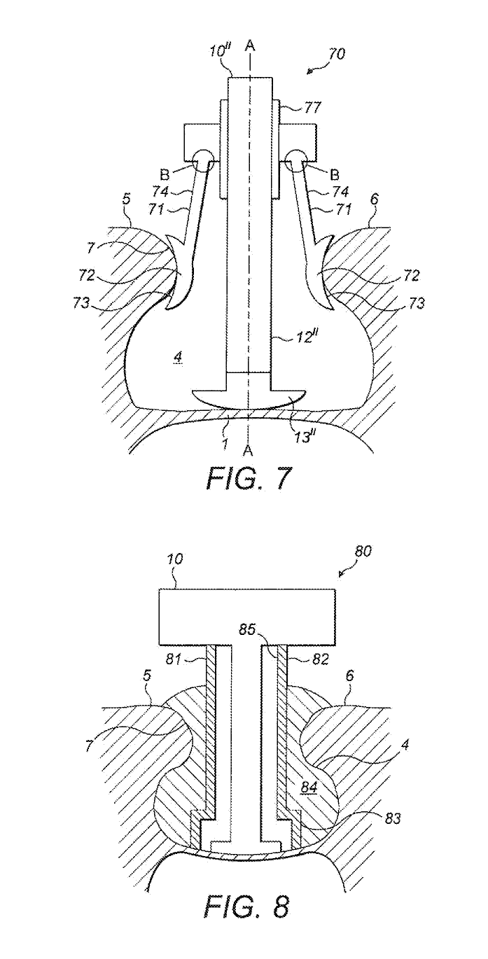

FIG. 7 illustrates a sixth embodiment of the present invention;

FIG. 8 illustrates a seventh embodiment of the present invention; and

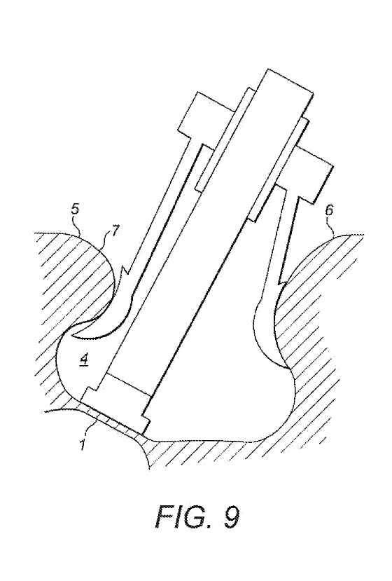

FIG. 9 illustrates an eighth embodiment of the present invention.

Components common to more than one figure or more than one embodiment are labelled in the figures using common reference numerals.

FIG. 1 shows the location of the round window 1 in the medial wall 2 of the tympanic cavity 3. The round window is located at the end of a funnel shaped depression known as the round window niche 4. The opening 7 of the niche is defined by bony prominences, labelled as 5 and 6, where 6 is the bony ridge of the subiculum. Although the bony prominences 5 and 6 are labelled as separate elements, these prominences extend into one another, to form a relatively narrow region at the opening of the round window niche, such that this niche is substantially funnel-shaped.

For the purposes of illustration, in FIGS. 2 to 8, the round window niche and the coupling apparatus located therein have been illustrated as if the round window membrane is located directly below the opening of the round window niche. However, it will be appreciated that the round window may be offset to one side of the niche, for example as illustrated in FIG. 9.

In accordance with a first embodiment of the invention, there is provided coupling apparatus in the form of a clip 20 for resiliently engaging the bony prominences 5, 6 which define the opening of the round window niche 4. The clip 20 is illustrated in FIG. 2.

The clip 20 is formed to be super-elastic, and comprises a pair of engagement or clamping arms 21 which extend in opposite directions from the centre of the clip.

One end of each of these arms 21 is shaped to form a curved section 22 for pressing against or gripping the region of bony prominences which define the opening of the round window niche. The concave surfaces 23 of these curved sections face in opposite directions, outwardly of the clip.

At the end of the curved sections 22, the clamping arms 21 curve away from the curved sections, through approximately 180 degrees, to form substantially semicircular sections 24. These two semicircular sections are connected at the centre of the clip by a curved connecting section 25 whose concave surface faces outwardly of the clip.

The two semicircular sections 24 and curved connecting section 25 form a connecting spring section 26, which connects the curved sections 22 of the clamping arms 21 and allows the clip to be resiliently deformed to pass through the opening 7 of the round window niche 4.

The clip 20 is configured such that, in its non-deformed state, the minimum width between the outwardly facing concave surfaces 23 of the clamping arms in a direction perpendicular to a longitudinal axis A of the clip is longer than the perpendicular distance between respective locations on opposite sides of the round window niche 4. This allows the clamping arms to press against the bone surface at the opening 7 of the niche, when inserted therethrough. At the same time, the clip is configured such that it can be resiliently deformed sufficiently for the clamping arms to fit through the opening.

The regions of the clip 20 which are deflected most when deforming the clip (ie, where the stress and strain are greatest) are identified by circles B in FIG. 2. The clip is configured such that these regions are deflected sufficiently from their original configuration when the clip is mounted to the bone surface within the round window niche, to cause the clip to operate in the super-elastic mode, such that the force exerted by the clamping arms 21 is substantially constant over a wide range of deformation.

The clip 20 further comprises an integrally formed mounting bracket 27 for supporting a hearing actuator 8.

The mounting bracket 27 comprises a generally L-shaped section which extends from the end of the curved section 22 of one of the clamping arms 21. In this respect, the mounting bracket comprises a first straight section 28 which extends from the end of the curved section, substantially parallel to the longitudinal axis A of the clip, and a second straight section 29 which extends from the end of the second straight section, substantially perpendicular to the longitudinal axis, to be intersected by this axis.

The hearing actuator 10 comprises a broad portion 11 for housing for a transducer (not shown), and an elongate portion 12 which extends from the broad portion. A plate 13 is formed at the end of the elongate portion, for contacting the round window membrane 1.

The broad portion 11 of the actuator 10 is bonded or otherwise mounted to an inward facing surface of the second straight section 29 of the mounting bracket 27, such that the elongate portion 12 extends between the clamping arms 21 and past the connecting spring section 26.

To implant the device shown in FIG. 2, a surgeon pinches together the two semi-circular sections 24 of the connecting spring section 26 using a surgical tool, such that the clamping arms 21 fit through the opening 7 of the round window niche 4. The device is then inserted through the opening to locate the clamping arms within the niche, with their respective curved sections 22 level with the bony prominences 5, 6 that define the opening. The surgeon then rotates the clip 20 to locate the curved sections at suitable locations on the bony prominences, and releases the clip. In this configuration, the mounting bracket 27 and the broad portion 11 of the actuator 10 are located outside the round window niche, whilst the elongate portion 12 of the actuator extends through the opening such that the plate 13 lies in contact with the round window membrane 1.

In practice, the form and dimensions of the round window niche, and the location of the round window membrane within the niche vary from patient to patient. To accommodate this variation, the actuator may be selected from a range of actuators of different sizes and configurations, or may be adjustable to achieve the correct configuration. Further, the first and second straight sections 28 and 29 of the mounting bracket 27 may be oriented to achieve the correct angle of the actuator relative to the clip, to allow the plate 13 to lie in contact with the round window membrane 1.

When the clip 20 is released, the clamping arms 21 try to revert to their original positions, causing them to press against the bony prominences 5, 6 to hold the clip and the mounted actuator 10 in place.

Accordingly, the device can not slide further into or out of the niche. However, if it is desired to remove or relocate the device, this is readily achieved by pinching together the semicircular sections 24 of the connecting spring section 26 using a surgical tool, and withdrawing the device from the niche.

It will be appreciated that the shape and size of the round window niche 4 may vary significantly from patient to patient. However, the super-elastic nature of the material from which the clip 20 is formed, and its configuration, means that the clamping arms 21 exert a substantially constant force over a wide range of deformation. Accordingly, a clip of given dimensions can accommodate significant variations in the dimensions of the round window niche. In practice, clips may be made in a range of sizes, such that a suitable clip may be selected for a patient on the basis of a pre-surgery scan, or during the surgery itself.

In the embodiment of FIG. 2, the clip 20 comprises two clamping arms 21 which extend in opposite directions from the centre of the clip. However, in other embodiments, one or more additional clamping arms may be provided, to increase the stability of attachment. The clamping arms are preferably evenly distributed around the centre of the clip. Accordingly, where there are three arms, these are preferably separated by angles of approximately 120 degrees, and where there are four arms, these are preferably separated by angles of approximately 90 degrees.

FIG. 3 illustrates a second embodiment of the present invention, in the form of a clip 30. The clip is again formed to be super-elastic, and comprises a pair of clamping arms 31 which extend in opposite directions from the centre of the clip.

One end of each of these arms 31 is shaped to form a curved section 32 for gripping the bony prominences 5, 6 at the opening 7 of the round window niche 4. The concave surfaces 33 of these curved sections face in opposite directions, outwardly of the clip.

At the end of the curved sections 32, the clamping arms 31 each comprise a straight section 34 which extends parallel to the longitudinal axis A of the clip. These two straight sections are connected by a third straight section 39, which extends perpendicularly to the longitudinal axis of the clip, and is intersected by this axis.

The clip 30 is configured such that, in its non-deformed state, the minimum width between the outwardly facing concave surfaces 33 of the clamping arms 31 in a direction perpendicular to the longitudinal axis A of the clip, is longer than the perpendicular distance between respective locations on opposite sides of the round window niche 4. This allows the clamping arms to press against the bone surface at the opening 7 of the niche, when inserted therethrough. At the same time, the clip is configured such that it can be resiliently deformed sufficiently for the clamping arms to fit through the opening.

The regions of the clip 30 which are deflected most when deforming the clip are identified by circles B in FIG. 3. The clip is configured such that these regions are deflected sufficiently from their original configuration when the clip is mounted to the bone surface within the round window niche, to cause the clip to operate in the super-elastic mode, such that the force exerted by the clamping arms 31 is substantially constant over a wide range of deformation.

The third straight section 39 acts as a mounting bracket 37 for a hearing actuator 10 similar to the actuator described in relation to FIG. 1.

The broad portion 11 of the actuator 10 is bonded or otherwise mounted to an inward facing surface of the third straight section 39 of the clip 30, such that the elongate portion 12 of the actuator extends between the curved sections 32 of the clamping arms 31.

To implant the device of FIG. 3, a surgeon pinches together the first and second straight sections 34 of the clip 30 using a surgical tool, to move the clamping arms 31 inwardly, as shown in dotted outline. In this configuration, the curved sections of the clamping arms fit through the opening 7 of the round window niche 4. The surgeon then inserts the device through the opening to align the curved sections of the clamping arms with the bony prominences 5, 6 which define the opening. The surgeon then rotates the clip to locate the curved sections at suitable locations on the bony prominences, and releases the clip. In this configuration, the mounting bracket 37 of the clip, and the broad portion 11 of the actuator 10 are located outside the niche, whilst the elongate portion 12 of the actuator extends through its opening, such that the plate 13 lies in contact with the round window membrane 1.

Again, the actuator may be selected from a range of actuators, or the actuator may be adjustable to accommodate variation in the form and dimensions of the round window niche. Further, the mounting bracket 37 may be oriented to achieve the correct angle of the actuator relative to the clip, to allow the plate 13 to lie in contact with the round window membrane 1.

When the clip is released, the clamping arms 31 try to revert to their original positions, such that the curved sections 32 press against the bony prominences 5, 6 to hold the clip in position.

Accordingly, the device can not slide further into or out of the niche. However, if it is desired to remove or relocate the device, this is readily achieved by pinching together the straight sections 34 of the clamping arms 31 using a surgical tool, and withdrawing the device from the niche.

Again, the super-elastic nature of the material from which the clip 30 is formed, and its configuration, means that the clamping arms 31 exert a substantially constant force over a wide range of deformation. Accordingly, a clip of given dimensions can accommodate significant variations in the dimensions of the round window niche.

For the arrangement shown in FIG. 3, the regions of maximum deflection are those identified by circles B. However, if a similar clip were used with a narrower actuator, the regions of maximum deflection may be those identified by circles C. In this case, these regions would be rounded rather than sharp.

Although the embodiment of FIG. 3 comprises two clamping arms 31 which extend in opposite directions from the centre of the clip, in other embodiments, one or more additional clamping arms may be provided, to increase the stability of attachment.

FIG. 4 illustrates a third embodiment of the present invention, in the form of a clip 40. The clip comprises a super-elastic collar 41 for location around the opening 7 of the round window niche 4. The collar is a split collar. That is to say, it has an incomplete, substantially circular form. The opposite ends 42 of the collar are separated by a variable distance, such that these can be brought together and moved apart to respectively decrease and increase the diameter of the collar.

The collar 41 has a substantially constant c-shaped cross section along its circular axis, to provide an outwardly facing concave surface 43 for engaging the bony prominences 5, 6 of the round window niche 7.

The clip 40 is configured such that the smallest diameter of the concave surface 43 is larger than the average diameter of the opening 7 of the round window niche 4. This allows the collar 41 to press against the bone surface at the opening of the niche, when inserted therethrough. At the same time, the clip is configured such that it can be resiliently deformed sufficiently for the collar to fit through the opening.

To implant the clip 40 of FIG. 4, a surgeon pinches the sides of the collar 41 together using a surgical tool, to bring the ends 42 of the collar closer together, and thereby reduce the diameter of the collar. In this configuration, the collar fits through the opening 7 of the round window niche 4. The surgeon then inserts the collar through the opening to align the collar with the bony prominences 5, 6 which define the opening, and releases the clip.

The clip 40 of FIG. 4 may be mounted directly to the broad portion 10 of an actuator such as the actuator 10 shown in FIGS. 1 and 2, or a mounting bracket may be provided for receiving the actuator, such that an elongate portion 12 of the actuator extends through the opening 44 defined by the collar, to contact the round window 1 when the clip is located within the round window niche 4.

FIG. 5 illustrates a fourth embodiment of the present invention in the form of a clip 50. The clip is again formed to be super-elastic.

The clip 50 comprises first and second straight sections 58 which extend parallel to the longitudinal axis A of the device, and which are joined at respective ends thereof by a third straight section 59 which extends perpendicular to the longitudinal axis of the clip. These three sections form a mounting bracket 57 for receiving the broad portion 11 of an actuator 10, similar to that shown in FIGS. 2 and 3.

Two clamping arms 51 extend from the other ends of the first and second straight sections 58 at an acute angle relative to the third straight section 59, to cross one another centrally of the clip 50.

The clamping arms 51 are, however, spaced apart, or angled away from the longitudinal axis A in a lateral direction, to leave a clear path along the longitudinal axis of the clip.

The tips 52 of the clamping arms are curved to form outwardly facing convex surfaces 53 for gripping the bone surface within the round window niche 4, on an underside of the bony prominences 5, 6 which define its opening 7.

The clip 50 is configured such that, in its non-deformed state, the maximum distance between the convex surfaces 53 at the end of the clamping arms 51, in a direction perpendicular to the longitudinal axis A of the clip, is longer than the perpendicular distance between respective locations on opposite sides of the round window niche 4. This allows the clamping arms to press against the bone surface of the niche, when inserted through the opening 7 of the niche. At the same time, the clip is configured such that the ends of the clamping arms can be resiliently brought towards one another to fit through the opening.

In particular, the first and second straight edges 58 are slightly longer than the corresponding dimension of the broad portion 11 of the actuator 10 in the longitudinal direction, such that when the actuator is mounted on the mounting bracket 57, the presence of the housing does not interfere with displacement of the clamping arms 51.

The regions of the clip 50 which are deflected most when deforming the clip are identified by circles B in FIG. 5. The clip is configured such that these regions are deflected sufficiently from their original configuration when the clip is mounted to the bone surface within the round window niche, to cause the clip to operate in the super-elastic mode, such that the force exerted by the clamping arms 51 is substantially constant over a wide range of deformation.

The broad portion 11 of the actuator 10 is bonded or otherwise mounted to an inward facing surface of the third straight section 59 of the mounting bracket 57, such that the elongate portion 12 of the actuator extends along the longitudinal axis A of the clip, between the clamping arms 51.

To implant the device of FIG. 5, a surgeon pinches together the first and second clamping arms 51 using a surgical tool, to reduce the distance between their respective end sections 52, and increase their angle with respect to the third straight section 59 of the mounting bracket 57. The deformed configuration of the clip is shown in dotted outline in FIG. 5. In this configuration, the end sections of the clamping arms fit through the opening 7 of the round window niche 4. The surgeon then inserts the device through the opening, rotates the clip to locate the first and second end sections at suitable locations on the underside of the bony prominences 5, 6, and releases the clip. In this configuration, the mounting bracket 57 of the clip, and the broad portion 11 of the actuator 10 are located outside the niche, whilst the elongate portion 12 of the actuator extends through its opening, such that the plate 13 lies in contact with the round window membrane 1.

Again, the actuator may be selected from a range of actuators, or may be adjustable to accommodate variation in the form and dimensions of the round window niche 4. Further, the mounting bracket 57 may be oriented to achieve the correct angle of the actuator relative to the clip, to allow the plate 13 to lie in contact with the round window membrane 1.

When the clip 50 is released, the clamping arms 51 try to revert to their original positions, such that their curved end sections 52 press upwardly and outwardly against the bony prominences 5, 6 to hold the clip in position.

Accordingly, the device can not slide further into or out of the niche. However, if it is desired to remove or relocate the device, this is readily achieved by pinching together the clamping arms 51 using a surgical tool, and withdrawing the device from the niche.

Again, the super-elastic nature of the material from which the clip 50 is formed, and its configuration, means that the clamping arms 51 exert a substantially constant force over a wide range of deformation. Accordingly, a clip of given dimensions can accommodate significant variations in the dimensions of the round window niche.

For the arrangement shown in FIG. 5, the regions of maximum deflection are those identified by circles B. However, if a similar clip were used with a narrower actuator, the regions of maximum deflection may be those identified by circles C. In this case, these regions would be rounded rather than sharp.

Although the embodiment of FIG. 5 comprises two clamping arms 51 which extend in opposite directions from the centre of the clip, in other embodiments, one or more additional clamping arms may be provided, to increase the stability of attachment.

FIG. 6 illustrates a fifth embodiment of the present invention in the form of a clip 60. The clip is again formed to be super-elastic.

The clip 60 comprises a cylindrical mounting portion 67 for adjustably engaging a hearing actuator 10′. Three curved clamping arms 61, two of which are shown in FIG. 6, extend from one end of the cylindrical mounting portion, separated by angles of approximately 120 degrees.

The three clamping arms 61 each comprise curved sections 62 which define an outwardly facing concave surface 63 for gripping the bony prominences 5, 6 which define the opening 7 of the round window niche 4.

The regions of the clip 60 which are deflected most when deforming the clip are identified by circle B in FIG. 6. The clip is configured such that these regions are deflected sufficiently from their original configuration when the clip is mounted to the bone surface within the round window niche, to cause the clip to operate in the super-elastic mode, such that the force exerted by the clamping arms 61 is substantially constant over a wide range of deformation.

The broad portion 11′ of the actuator 10′ has a cylindrical form, with a diameter equal to the internal diameter of the cylindrical mounting portion 67. The external surface of the housing and the internal surface of the mounting portion are provided with correspondingly formed threads (not shown), such that the actuator can be releasably mounted to the clip 60 by screwing the broad portion into the cylindrical mounting portion. The longitudinal position of the actuator with respect to the clip can then be adjusted by rotating the actuator with respect to the clip.

To implant the device of FIG. 6, the surgeon first mounts the clip 60 to the bony prominences 5, 6 of the round window niche 4 by pinching together the clamping arms 61 using a surgical tool, inserting these through the opening 7 of the round window niche, rotating the clip into a suitable orientation, and releasing the clip. The surgeon then inserts the actuator through the cylindrical mounting portion 67, such that the elongate portion 12 of the actuator extends between the clamping arms 61 of the clip, and engages the threaded surfaces of the clip and the actuator. The surgeon then rotates the actuator with respect to the clip using a screwdriver or the like, to adjust the longitudinal position of the actuator until the plate 13 at the end of the elongate portion comes into contact with the round window 1.

The actuator is then maintained in this position through engagement of the clamping arms 61 with the bone surface within the round window niche 4, and through frictional engagement between the respective threaded sections of the broad portion 11′ of the actuator 10′ and the cylindrical mounting portion 67 of the clip 60.

Again, the super-elastic nature of the material from which the clip 60 is formed, and its configuration, means that the clamping arms 61 exert a substantially constant force over a wide range of deformation. Accordingly, a clip of given dimensions can accommodate significant variations in the dimensions of the round window niche.

Although the embodiment of FIG. 6 comprises three clamping arms, in other embodiments, the clip may comprise two clamping arms, or more than three clamping arms.

In an alternative embodiment, the correspondingly formed threads may be replaced by correspondingly formed recess and projections, such that the actuator may be slidably engaged by the mounting part of the clip.

In general, the adjustable mounting means of the fifth embodiment may be applied to any or all of the other embodiments described herein.

FIG. 7 illustrates a sixth embodiment of the present invention in the form of a clip 70, which is configured for engagement with an actuator 10″ having a substantially elongate cylindrical form. The clip is again formed to be super-elastic.

The clip 70 comprises a cylindrical mounting portion or sleeve 77 for adjustably engaging the hearing actuator 10″. The sleeve located within a frame 79. Two curved clamping arms 71 extend from opposite sides of the frame.

The clamping arms 71 each comprise a straight section 74, and a curved section 72. Each curved section defines an outwardly facing concave surface 73 for gripping the bony prominences 5, 6 which define the opening 7 of the round window niche 4.

The regions of the clip 70 which are deflected most when deforming the clip are identified by circles B in FIG. 7. The clip is configured such that these regions are deflected sufficiently from their original configuration when the clip is mounted to the bone surface within the round window niche, to cause the clip to operate in the super-elastic mode, such that the force exerted by the clamping arms 71 is substantially constant over a wide range of deformation.

The actuator 10″ has an elongate portion 12″, which forms a housing for an elongate transducer (not shown), and a plate 13″ at one end of the elongate portion. The elongate portion has a substantially cylindrical form, configured to slidably engage with the sleeve 77. Accordingly, the longitudinal position of the actuator with respect to the clip can then be adjusted by sliding the actuator with respect to the clip. The clip is then held in position relative to the clip through friction between the respective surfaces of the actuator and the clip. Alternatively, clamping means may be provided for holding the actuator in position, once it is located in a desired position.

To implant the device of FIG. 7, the actuator 10″ is located in the clip, such that the elongate portion 12″ extends through the sleeve 77, such that the plate 13″ abuts the end of the sleeve. A surgeon pinches together the first and second clamping arms 71 using a surgical tool, to reduce the distance between the curved sections 72. In this configuration, the curved sections of the clamping arms fit through the opening 7 of the round window niche 4. The surgeon then inserts the device through the opening, rotates the clip to locate the first and second end sections at suitable locations on the underside of the bony prominences 5, 6, and releases the clip. In this configuration, the sleeve 77 of the clip, and the actuator are located outside the niche. The surgeon then presses on the exposed end of the actuator, in order to slide the actuator relative to the sleeve, through the opening of the round window niche, until the plate 13′ rests against the round window membrane 1. The surgeon then clamps the actuator in position.

Again, the super-elastic nature of the material from which the clip 70 is formed, and its configuration, means that the clamping arms 71 exert a substantially constant force over a wide range of deformation. Accordingly, a clip of given dimensions can accommodate significant variations in the dimensions of the round window niche.

Although the embodiment of FIG. 7 comprises two clamping arms 71 which extend in opposite directions from the centre of the clip, in other embodiments, one or more additional clamping arms may be provided, to increase the stability of attachment.

In general, the adjustable mounting means of the sixth embodiment may be applied to any or all of the other embodiments described herein.

In further embodiments of the invention, the orientation of the actuator or other implant relative to the clip can be adjusted by means of a releasably clampable ball and socket joint.

The above described embodiments of the invention each comprise two or more resiliently deformable or movable clamping arms. However, in general, provided the coupling apparatus comprises at least one resiliently deformable or movable engagement section, the remaining engagement sections may be substantially rigid.

FIG. 8 illustrates a seventh embodiment of the present invention.

In the seventh embodiment, the coupling apparatus 70 comprises a tubular member 81 for location within the round window niche 4, and a filler material 82 for securing the tubular member in position within the round window niche. The filler material may be an ionic cement, hydroxyapatite or other biocompatible filling material.

The tubular member 81 has a first cylindrical section 82, and a second, wider cylindrical section 83 at one end, for location around the round window membrane 1, to form a seal. The diameter of both sections 82, 83 is smaller than that of the opening 7 of the round window niche 4, such that the tubular member can be inserted through this opening to form a channel or passage 85 from outside the niche to the round window membrane.

To implant the device of FIG. 8, the tubular member 81 is inserted through the opening 7 of the round window niche 4, and the second cylindrical section 83 is located around the round window membrane 1, to form a seal therewith.

A filler material 84 is then injected into the round window niche 7 to surround the tubular member. The filler material is then allowed to set, such that the tubular member 81 is securely held in place.

The tubular member 81 thus forms a channel or passage 85 to the round window membrane 1, through which an actuator 10 or other implantable device can be inserted, for stimulating the round window membrane.

The actuator may be integrally formed with the tubular member 81, or may be fixedly or adjustably mounted thereto, by means of a mounting bracket, or adjustably mounting part, as described in relation to the previous embodiments.

For the purposes of illustration, in FIGS. 2 to 8, the round window niche and the coupling apparatus located therein have been illustrated as if the round window membrane is located directly below the opening of the round window niche. In general, the clips illustrated in FIGS. 2 to 7 and can accommodate different anatomical variations in which, for example, the round window membrane is offset to the side of the niche. However, in some cases, it may be desirable to specifically configure the clip to accommodate a particular variation. This can be achieved by varying the form of the clip as illustrated in FIG. 9. It will be appreciated that similar variations may be applied to any of the clips illustrated in FIGS. 2 to 7.

The present invention has been described in terms of coupling apparatus for coupling a hearing actuator to the round window membrane. However, it will be appreciated that the principles of the invention may be applied to middle ear implants of other types, including passive implants such as prostheses. In particular, the principles of the invention may be applied to implants which extend to the round window from other parts of the middle ear, such as a point on the ossicular chain, the temporal bone, or a point outside the middle ear.

1-33. (canceled)

34. Coupling apparatus for coupling an implantable element to the round window membrane, the apparatus comprising engagement means for engaging the bone surface within the round window niche, to support the apparatus in the region of the round window membrane, wherein said engagement means is at least partially formed of a super-elastic material, and wherein said engagement means is super-elastically deformable between a first configuration for insertion through the opening of the round window niche, and a second configuration for engaging the bone surface within the niche.

35. Coupling apparatus according to claim 34 wherein the engagement means is configured for engaging location(s) on the bony prominences which define the round window niche.

36. Coupling apparatus according to claim 34 wherein the material from which the clip is formed is a nickel titanium alloy.

37. Coupling apparatus according to claim 34 wherein the engagement means comprises a single engagement section.

38. Coupling apparatus according to claim 34 wherein the single engagement means takes the form of a split collar.

39. Coupling apparatus according to claim 34 wherein the engagement means comprises a plurality of engagement sections for engaging respective locations on the bone surface within the round window niche.

40. Coupling apparatus according to claim 39 wherein the engagement sections are substantially evenly distributed about a longitudinal axis of the apparatus.

41. Coupling apparatus according to claim 39 wherein the coupling apparatus comprises two engagement sections separated by angles of approximately 180 degrees.

42. Coupling apparatus according to claim 39 wherein the coupling apparatus comprises three engagement sections separated by angles of approximately 120 degrees.

43. Coupling apparatus according to claim 39 wherein the engagement sections each take the form of an engagement arm which extends from a common connecting portion.

44. Coupling apparatus according to claim 43 wherein the connecting portion is positioned for location externally of the round window niche when the engagement means is engaged with the bone surface within the niche.

45. Coupling apparatus according to claim 37 wherein the or each engagement section comprises a curved section which forms an outwardly facing concave surface for engaging the bone surface of the round window niche.

46. Coupling apparatus for coupling an implantable element to the round window membrane, the apparatus comprising engagement means for engaging the bone surface within the round window niche to support the apparatus in the region of the round window membrane, wherein the coupling apparatus comprises a tubular portion for location over or around the round window membrane, wherein the engagement means comprises a filler material for at least partially filling the space between an external surface of the tubular portion and the bone surface within the round window niche, to engage said bone surface and hold the tubular portion in position in relation to the round window membrane.

47. Coupling apparatus according to claim 46 wherein the filler material is one of an ionic cement, hydroxyapatite, or other biocompatible filling material.

48. Coupling apparatus according to claim 34 further comprising a mounting part for mounting to an implantable element.

49. Coupling apparatus according to claim 48 wherein the mounting part of the coupling apparatus is positioned for location externally of the round window niche when the engagement means is engaged with the bone surface within the round window niche.

50. Coupling apparatus according to claim 48 wherein the mounting part comprises adjustable mounting means for mounting to an implantable element, such that the mounted position of the implantable element in relation to the coupling apparatus is adjustable.

51. Coupling apparatus according to claim 34 including an implantable element, which forms an integral part of the coupling apparatus, or is mounted to a mounting part of the apparatus.

52. Coupling apparatus according to claim 51 wherein the coupling apparatus is configured such that, when the engagement means is engaged with the bone surface within the round window niche, the implantable element extends to or towards the round window membrane, for conveying vibrational energy thereto.

53. Coupling apparatus according to claim 52 wherein the implantable element extends through the opening of the round window niche.

54. A method of coupling an implantable element to the round window membrane, the method comprising:
providing coupling apparatus for coupling a hearing actuator to the round window membrane; and
engaging said apparatus with the bone surface within the round window niche.

55. A method according to claim 54 further comprising:
deforming a resiliently deformable engagement means of the coupling apparatus into a first configuration;
inserting the engagement means through the opening of the round window niche in said first configuration; and
releasing the engagement means such that the engagement means engages the bone surface within the round window niche.

56. A method according to claim 54 further comprising:
locating a tubular member over or around the round window membrane;
at least partially filling a space between the external surface of the tubular member and the bone surface within the round window niche with a filler material; and
allowing the filler material to set to hold the tubular member in place.
